(19) United States
(12) Patent Application Publication
(10) Pub. No.: US 2016/0151588 A1
(43) Pub. Date: Jun. 2, 2016

(54) Pharmaceutical Metered Dose Inhaler and Methods Relating Thereto

(71) Applicant: Glaxo Group Limited, Brentford (GB)

(72) Inventors: John Francis MILLER, Durham, NC (US); Mark Lee SOMMERVILLE, Durham, NC (US); Robert David SCHULTZ, Durham, NC (US)

(21) Appl. No.: 15/016,421

(22) Filed: Feb. 5, 2016

Related U.S. Application Data
(63) Continuation of application No. 10/570,033, filed on Feb. 28, 2006.
(60) Provisional application No. 60/499,250, filed on Aug. 29, 2003.

(51) Int. Cl. A61M 15/00; A61K 31/137; A61K 31/569; A61M 16/20

(52) U.S. Cl. CPC A61M 15/009; A61M 15/0025; A61M 16/20; A61K 31/137; A61K 31/569

(57) ABSTRACT

Metering valves for use in a metered dose inhaler that include a valve body, a first stem seal including a first elastomeric material, a second stem seal including a second elastomeric material different from the first elastomeric material, and a valve stem slidably engaged with at least one of the first stem seal and the second stem seal as well as sealed containers configured to contain an aerosol pharmaceutical formulation that include a container having an opening therein, a cap covering the opening in the container, a metering valve adjacent the cap, and a cap seal positioned between the cap and the container to provide a sealed container where the metering valve include at least one stem seal that includes a first elastomeric material, and the cap seal includes a second elastomeric material different from the first elastomeric material are described.

### FIELD OF THE INVENTION

The present invention relates to medical devices as well as methods of making and using same. The medical devices are useful in the treatment of respiratory or other disorders.

### BACKGROUND OF THE INVENTION

The use of aerosols to administer medicaments has been known for several decades. Such aerosols generally comprise one or more medicaments, one or more propellants and optionally one or more additives, for example a surfactant or a co-solvent, such as ethanol. Historically the most commonly used aerosol propellants for medicaments have been propellant 11 (CCl₃F), propellant 114 (CF₂ClCF₂Cl), propellant 12 (CCl₂F₂) or combinations of those. However release of those propellants into the atmosphere is now believed to contribute to the degradation of stratospheric ozone and there is thus a need to provide aerosol formulations for medicaments which employ so called “ozone-friendly” propellants.

Containers for aerosol formulations commonly comprise a vial body (e.g., can or canister) coupled to a valve. The valve comprises a valve stem through which the formulations are dispensed. Generally the valve includes one or more rubber valve seals intended to allow reciprocal movement of the valve stem which prevents leakage of propellant from the container Metered dose inhalers comprise a valve which is designed to deliver a metered amount of an aerosol formulation to the recipient per actuation. Such a metering valve generally comprises a metering chamber which is of a pre-determined volume and which causes the dose per actuation to be an accurate, pre-determined amount.

The metering valve in a container is typically coupled to the canister with contact through a sealing gasket to prevent leakage of propellant and/or drug substance out of the container at the join. The gasket typically comprises an elastomeric material, for example low density polyethylene, chlorobutyl, acrylonitrile butadiene rubbers, butyl rubber, a polymer of ethylene propylene diene monomer (EPDM), neoprene or chloroprene. Such elastomeric materials may be carbon-black or mineral filled. Valves for use in MDIs are available from various manufactures known in the aerosol industry; for example from Valois, France (e.g. DF10, DF30, DF60), Bespak plc, UK (e.g. BK300, BK356, BK357) or 3M-Neotechnic Limited, UK (e.g. Spraymiser™). The metering valves are used in association with commercially available canisters, for example metal canisters, for example aluminium canisters, suitable for delivering pharmaceutical aerosol formulations.

MDIs incorporating a valve seal or a sealing gasket as described above generally perform adequately with CFC propellants, such as propellant 11 (CCl₃F), propellant 114 (CF₂ClCF₂Cl), propellant 12 (CCl₂F₂). However, as mentioned above, there is a requirement to substitute so-called ozone-friendly propellants for CFC propellants in aerosols. A class of propellants which are believed to have minimal ozone-depleting effects in comparison to conventional chlorofluorocarbons comprise fluorocarbons and hydrogen-containing chlorofluorocarbons. That class includes, but is not limited to hydrofluoroalkanes (HFAs), for example 1,1,1,2-tetrafluoroethane (HFA134a), 1,1,1,2,3,3,3-heptafluoro-n-propane (HFA 227) and mixtures thereof. However, various problems have arisen with pharmaceutical aerosol formulations prepared using HFA propellants, in particular with regard to the stability of the formulations.

Pharmaceutical aerosol formulations generally comprise a solution or a suspension. A mixture of a suspension and a small amount of dissolved medicament is also possible, but generally undesirable (as described below). Some solution formulations have the disadvantage that the drug substance contained therein is more susceptible to degradation than when in solid form. Furthermore, solution formulations may be associated with problems in controlling the size of the droplets which in turn affects the therapeutic profile. Suspension formulations are thus generally preferred.

To obtain regulatory approval, pharmaceutical aerosol formulation products must satisfy strict specifications. One parameter that must generally be satisfied, and for which a level is usually specified, is the fine particle mass (FPM). The FPM is a measure of the amount of drug that has the potential to reach the inner lungs (the small bronchioles and alveoli) based on the proportion of drug particles with a diameter within a certain range, usually less than 5 microns. The FPM of an actuation from an MDI is generally calculated on the basis of the sum of the amount of drug substance deposited on stages 3, 4 and 5 of an Andersen Cascade Impaction stack as determined by standard HPLC analysis. Potential side effects are minimised and a smaller amount of drug substance is wasted if the FPM constitutes as large as possible a percentage of the total mass of drug.

In suspension formulations, particle size of the emitted dose is generally controlled during manufacture by the size to which the solid medicament is reduced, usually by micronisation. During storage of some drug suspensions in an HFA, however, various changes have been found to take place which have the effect of reducing FPM. A drop in FPM means that the therapeutically effective amount of drug available to the patient is reduced. That is undesirable and may ultimately impact on the effectiveness of the medication. That problem is particularly acute when the dose due to be dispensed is low, which is the case for certain potent drugs such as long acting beta agonists, which are bronchodilators.

Various mechanisms have been proposed by which the reduction in FPM may be taking place: particle size growth may occur if the suspended drug has a sufficient solubility in propellant, a process known as Ostwald Ripening. Alternatively, or additionally, small particles may have the tendency to aggregate or adhere to parts of the inside of the MDI, for example the canister or valve Small particles may also become absorbed into or adsorbed onto rubber components of the valve. As adherence and absorption processes are more prevalent amongst small particles, those processes may lead to a decrease in FPM as a fraction of the administered drug as well as a reduction in the total drug content (TDC) of the canister available to patient. It has further been found that the adherence and absorption processes may not only result in loss of available drug, but may also adversely affect the function of the device, resulting in the valve sticking or orifices becoming blocked.

It is essential that the prescribed dose of aerosol medication delivered from the MDI to the patient consistently meets the specifications claimed by the manufacturer and complies with the requirements of the FDA and other regulatory authorities. That is, every dose dispensed from the MDI must be the same within close tolerances. Therefore it is important that the formulation be substantially homogenous throughout the canister and the administered dose at the time of actuation of the metering valve and that it remains substantially the same even after storage.

Various approaches have been taken to address the problems mentioned above. One approach is the addition of one or more adjuvants to the drug suspension; for example adjuvants selected from alcohols, alkanes, dimethyl ether, surfactants (e.g. fluorinated or non-fluorinated surfactants, carboxylic acids, polyethoxylates, etc.) and even conventional chlorofluorocarbon propellants in small amounts (at levels intended to keep to a minimum potential ozone damage) have been shown to have some effect in mitigating the FPM problems. Such approaches have been disclosed, for example, in EP0372777, WO91/04011, WO91/11173, WO91/11495 and WO91/14422. WO92/00061 discloses non-fluorinated surfactants for use with fluorocarbon propellants. Fluorinated surfactants may be used to stabilise micronised drug suspensions in fluorocarbon propellants such as 1,1,1,2-tetrafluoroethane (P134a) or 1,1,1,2,3,3,3-heptafluoro-n-propane (P227), see for example U.S. Pat. No. 4,352,789, U.S. Pat. No. 5,126,123, U.S. Pat. No. 5,376,359, U.S. application Ser. No. 09/580,008, WO91/11173, WO91/14422, WO92/00062 and WO96/09816.

In WO96/32345, WO96/32151, WO96/32150 and WO96/32099 there are disclosed aerosol canisters coated with one or more fluorocarbon polymers, optionally in combination with one or more non-fluorocarbon polymers, that reduce the deposition on the canister walls of drug particles of the pharmaceutical alternative propellant aerosol formulation contained therein.

In WO 03/049786 it is described that deposition of drug on an elastomeric seal, and several other problems associated with lubrication, flexibility and sealing ability of an elastomeric seal may be overcome by the addition of an organotitanium low friction barrier coating to the seal surface. A pre-treatment step in which the elastomeric seal is treated as follows is also disclosed therein: the elastomeric substrate is provided in a bath comprising an alcohol and an alkaline material at a bath temperature effective for treatment, ultrasonic energy is provided to the bath at a treatment effective frequency and power level for a time sufficient to treat the elastomeric substrate, the treated elastomeric substrate is rinsed with de-ionised water; and the treated and rinsed elastomeric substrate is dried. The pre-treatment step is said to permit superior adhesion and bonding of the organotitanium-based coating In general, however, additional material coating steps add to the expense of manufacturing the final drug product and the presence of a coating may cause additional toxicity and safety tests to be necessary.

The present invention is concerned with medical devices and portions thereof, such as metered dose inhalers and/or metering valves, that may provide improved stability of pharmaceutical formulations contained therein.

### SUMMARY OF THE INVENTION

Applicants have surprisingly discovered that when the neck (or cap) seal of a metered dose inhaler (MDI) comprises a different elastomeric material than the material used for a stem seal in the metering valve of the (MDI), a pharmaceutical formulation contained in the MDI can exhibit an improved stability (e.g., a decreased drop in FPM after storage) compared to an MDI in which the cap seal and the stem seal comprise the same elastomeric material Similar beneficial results may be observed when the metering valve of a conventional MDI possesses two stem seals comprising different materials.

According to embodiments of the present invention, a metering valve for use in a metered dose inhaler includes a valve body, a first stem seal including a first elastomeric material, a second stem seal including a second elastomeric material different from the first elastomeric material, and a valve stem slidably engaged with at least one of the first stem seal and the second stem seal. In some embodiments, the valve body and the first stem seal and/or the second stem seal define a metering chamber.

According to other embodiments of the present invention, a method of making a metering valve for use in a metered dose inhaler includes assembling a valve body, a first stem seal that includes a first elastomeric material, a second stem seal that includes a second elastomeric material different from the first elastomeric material, and a valve stem to provide a metering valve.

According to still other embodiments of the present invention, a sealed container configured to contain an aerosol pharmaceutical composition includes a container having an opening therein, a cap covering the opening in the container, a metering valve adjacent the cap, and a cap seal positioned between the cap and the container to provide a sealed container configured to contain an aerosol pharmaceutical composition. The metering valve includes at least one stem seal that includes a first elastomeric material, and the cap seal includes a second elastomeric material different from the first elastomeric material.

According to yet other embodiments of the present invention, a method for making a sealed container configured to contain an aerosol pharmaceutical composition includes assembling a container having an opening therein, a cap configured to cover the opening in the container, a metering valve including at least one stem seal that includes a first elastomeric material, and a cap seal that includes a second elastomeric material different from the first elastomeric material to provide the sealed container configured to contain an aerosol pharmaceutical composition.

According to other embodiments of the present invention, a medicament dispenser includes a sealed container that includes a container having an opening therein, a cap covering the opening in the container, a metering valve adjacent the cap, and a cap seal positioned between the cap and the container to provide a sealed container configured to contain an aerosol pharmaceutical composition, and an aerosol pharmaceutical composition contained within the sealed container The metering valve includes at least one stem seal that includes a first elastomeric material and the cap seal includes a second elastomeric material different from the first elastomeric material.

According to still other embodiments of the present invention, a method of making a medicament dispenser includes filling a sealed container that includes a container having an opening therein, a cap configured to cover the opening in the container, a metering valve, and a cap seal with an aerosol pharmaceutical formulation to provide a medicament dispenser. The metering valve includes at least one stem seal that includes a first elastomeric material and the cap seal includes a second elastomeric material different from the first elastomeric material.

According to yet other embodiments of the present invention, a metered dose inhaler includes a medicament dispenser according to embodiments of the present invention, and an actuator engaging the medicament dispenser and configured to dispense the pharmaceutical composition from the medicament dispenser.

According to other embodiments of the present invention, a method of making a metered dose inhaler includes assembling a medicament dispenser that includes a container having an opening therein, a cap configured to cover the opening in the container, a metering valve, a cap seal comprising a second elastomeric material, and a pharmaceutical composition contained within the container, with an actuator configured to engage the medicament dispenser and dispense a pharmaceutical composition therefrom to provide the metered dose inhaler The metering valve includes at least one stem seal that includes a first elastomeric material and the cap seal includes a second elastomeric material different from the first elastomeric material.

According to still other embodiments of the present invention, a drug product includes a metered dose inhaler according to embodiments of the present invention and a packaging material forming an enclosed volume that contains the metered dose inhaler

According to yet other embodiments of the present invention, a method of making a drug product includes packaging a metered dose inhaler according to embodiments of the present invention within a packaging material to provide the drug product.

According to other embodiments of the present invention, a method of distributing a sealed container includes transporting a sealed container according to embodiments of the present invention over a distance of at least 1 mile.

According to still other embodiments of the present invention, a method of administering a pharmaceutical composition comprising a medicament indicated for the treatment of a respiratory disease, or other disease or condition, to a subject in need thereof includes actuating a metered dose inhaler according to embodiments of the present invention to administer the pharmaceutical composition to the subject.

### BRIEF DESCRIPTION OF THE DRAWINGS

FIG. 1 illustrates a sealed container according to embodiments of the present invention;

FIG. 2 illustrates a sectional view taken along the line I-I of a portion of the sealed container illustrated in FIG. 1;

FIG. 3 illustrates a sectional view of a portion of a sealed container according to embodiments of the present invention; and

FIG. 4 illustrates a metered dose inhaler according to embodiments of the present invention.

### DESCRIPTION OF PREFERRED EMBODIMENTS OF THE PRESENT INVENTION

The invention will now be described with respect to preferred embodiments described herein. It should be appreciated however that these embodiments are for the purpose of illustrating the invention, and are not to be construed as limiting the scope of the invention as defined by the claims. Like reference numerals refer to like elements throughout.

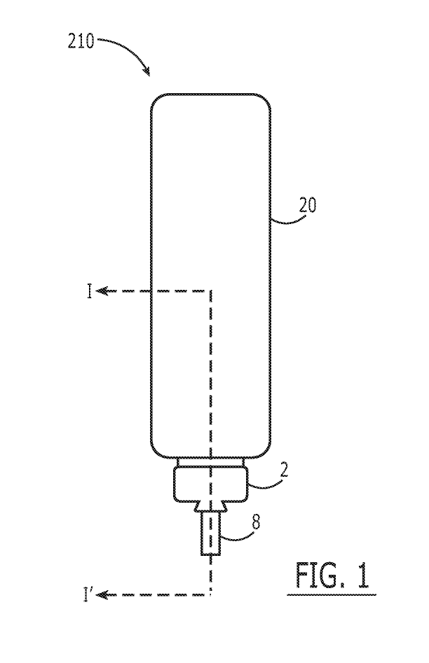

Referring first to FIG. 1, a sealed container 210 according to embodiments of the present invention will be described. The sealed container 210 includes a container 20. While the container 20 as illustrated in FIG. 1 is in the shape of a can or canister, it will be understood by those skilled in the art that the container 20 can have various other shapes including, but not limited to, spherical and oblong. The container 20 may be made of various materials as will be understood by those skilled in the art including, but not limited to, plastics, plastics-coated glass, and metal. The metal may be various metals as will be understood in the art including, but not limited to, aluminum and stainless steel. The metal is preferably aluminium or an alloy thereof which may optionally be anodised, lacquer-coated and/or plastic-coated (e.g., as described in U.S. Pat. Nos. 6,131,566, 15 6,143,277, 6,149,892, 6,253,762, 6,511,652, 6,511,653, 6,524,555, 6,532,955, and 6,546,928 wherein part or all of the internal surfaces of the can are coated with one or more fluorocarbon polymers optionally in combination with one or more non-fluorocarbon polymers). When the sealed container 210 is used to contain an aerosol pharmaceutical formulation, for example in a metered dose inhaler application, the container is preferably made of a material capable of withstanding the vapour pressure of the propellant used. Such materials include plastics, plastics-coated glass, and metal materials as described above.

The container 20 has an opening therein with a cap 2 covering the opening in the container 20. A metering valve having a valve stem is positioned within the sealed container 210. A portion 8 of the valve stem protrudes from the cap 2. The cap 2 may be made of various materials as will be understood in the art including, but not limited to, plastic and metal. The cap is preferably made of a metal material such as stainless steel, aluminum or an aluminum alloy. The cap may be secured onto the canister via welding such as ultrasonic welding or laser welding, screw fitting or crimping. Preferably the container 20 is fitted with a cap assembly, wherein a metering valve is situated in the cap 2, and the cap 2 is crimped in place.

According to embodiments of the present invention, a method for making a sealed container configured to contain an aerosol pharmaceutical formulation includes assembling a container having an opening therein, a cap configured to cover the opening in the container, a metering valve including at least one stem seal that includes a first elastomeric material, and a cap seal that includes a second elastomeric material different from the first elastomeric material to provide the sealed container configured to contain an aerosol pharmaceutical composition. In some embodiments, the assembling operation comprises providing a cap assembly that includes the metering valve coupled to the cap, and coupling the cap assembly to the container such that the metering valve is positioned within the container, the cap seal is positioned between the cap and the container, and the cap covers the opening of the container The cap assembly may be coupled to the container by various processes as will be understood by those skilled in the art including, but not limited to, welding such as ultrasonic welding or laser welding, screw fitting or crimping. In some embodiments, the cap assembly is provided by coupling the metering valve to the cap. The coupling of the metering valve to the cap may be performed by various processed including, but not limited to, crimping the valve into the cap.

In some embodiments, a medicament dispenser is provided. The medicament dispenser includes a sealed container according to the present invention, such as the sealed container 210, that contains a pharmaceutical formulation. The pharmaceutical formulation is preferably an aerosol pharmaceutical formulation (e.g., a formulation that is present in the liquid and/or gaseous phase when contained in the container, but is delivered as an aerosol to the patient). The pharmaceutical formulation may comprise one or more medicaments that may be administered in aerosol formulations and/or are useful in inhalation therapy including, but not limited to, analgesics, e.g. codeine, dihydromorphine, ergotamine, fentanyl or morphine; anginal preparations, e.g. diltiazem; anti-allergics, e.g. cromoglycate (e.g. as the sodium salt), ketotifen or nedocromil (e.g. as sodium salt); antiinfectives e.g. cephalosporin, penicillins, streptomycin, sulphonamides, tetracyclines and pentamidine; antihistamines, e.g. methapyrilene; anti-inflammatories, such as anti-inflammatory steroids, e.g. beclomethasone (e.g. as dipropionate), fluticasone (e.g. as propionate), flunisolide, budesonide, tipredane, rofleponide, mometasone (e.g as furoate), ciclesonide, triamcinolone acetonide, or 6α,9α-difluoro-11β-hydroxy-16α-methyl-3-oxo-17α-propionyloxy-androsta-1,4-diene-17β-carbothioic acid (e.g., ad the (2-oxo-tetrahydro-furan-3-yl)ester), or 6α,9α-difluoro-17α-[(2-furanylcarbonyl)oxy]-11β-hydroxy-16α-methyl-3-oxo-androsta-1,4-diene-17β-carbothioic acid (e.g., as the fluoromethyl ester); antitussives, e.g. noscapine; anticholinergics, e.g. ipratropium (e.g. as bromide), tiotropium, atropine or oxitropium; bronchodilators, e.g. albuterol (e.g., as free base or sulphate), salbutamol, salmeterol (e.g., as xinafoate), ephedrine, adrenaline, fenoterol (e.g., as hydrobromide), formoterol (e.g., as fumarate), isoprenaline, metaproterenol, phenylephrine, phenylpropanolamine, pirbuterol (e.g., as acetate), reproterol (e.g., as hydrochloride), rimiterol, terbutaline (e.g., as sulphate), isoetharine, tulobuterol, orciprenaline, 4-hydroxy-7-[2-[[2-[[3-(2-phenylethoxy)propyl]sulfonyl]ethyl]amino]ethyl-2(3H)-benzothiazolone, or (−)4-amino-3,5-dichloro-α-[[[6-[2-(2-pyridinyl)ethoxy]hexyl]amino]methyl]benzenemethanol; PDE4 inhibitors, e.g., cilomilast or roflumilast; leukotriene antagonists, e.g., montelukast, pranlukast or zafirlukast; diuretics, e.g. amiloride; hormones, e.g. cortisone, hydrocortisone or prednisolone; xanthines e.g. aminophylline, choline, theophyllinate, lysine theophyllinate or theophylline; and therapeutic proteins and peptides, e.g. insulin or glucagon. It will be clear to a person skilled in the art that, where appropriate, the one or more medicaments may be used in the form of salts, (e.g. as alkali metal or amine salts or as acid addition salts) or as esters (e.g. lower alkyl esters) or as solvates (e.g. hydrates). In some embodiments, the one or more medicaments may be used in the form of salts, esters, or solvates to optimise the activity and/or stability of the medicament and/or to minimise the solubility of the medicament in the propellant. Where applicable, the one or more medicaments may be used in the form of racemate (in equal or unequal proportions) or in the form of a pure isomer, e.g. R-salmeterol or S-salmeterol.

In some embodiments according to the present invention, the pharmaceutical formulation includes two or more of the medicaments described above, preferably 2, 3, or 4 of the medicaments described above, more preferably 2 or 3 of the medicaments described above, and still more preferably 2 of the medicaments described above. In preferred embodiments, the two or more medicaments are selected from the group consisting of a bronchodilator, an anti-inflammatory, an anticholinergic, and an antiallergic. In more preferred embodiments, the medicaments in the pharmaceutical formulation consist of a bronchodilator and an anti-inflammatory The bronchodilator is preferably salbutamol (e.g., as the free base or the sulphate salt), salmeterol (e.g., as the xinafoate salt), or formoterol (e.g., as the fumarate salt). The anti-inflammatory is preferably beclomethasone (e.g., as the dipropionate ester), fluticasone (e.g., as the propionate ester) or budesonide. Combinations of salmeterol xinafoate and fluticasone propionate or beclomethasone dipropionate, or salbutamol and fluticasone propionate or beclomethasone dipropionate are preferred, with salmeterol xinafoate and fluticasone priopionate or salbutamol and beclomethasone dipropionate being particularly preferred.

In some embodiments, the pharmaceutical formulation includes a combination of salmeterol xinafoate and fluticasone propionate and no further medicament substances are present.

The medicament is preferably present in the pharmaceutical formulation as a particulate medicament. The particle size of the particulate (e.g. micronised) medicament should be such as to permit inhalation of substantially all of the medicament into the lungs upon administration of the aerosol formulation and will thus be less than 100 microns, desirably less than 20 microns, and preferably in the range 1-10 microns, e.g. 1-5 microns.

The concentration of medicament in the formulation will generally be 0.01-10% such as 0.01-2%, particularly 0.01-1%, especially 0.03-0.25% w/w. When salmeterol xinafoate is the only medicament, its concentration in the formulation will generally be 0.03-0.15% w/w.

Aerosol pharmaceutical formulations according to embodiments of the present invention will include a propellant. The propellant may be selected from various propellants suitable for use in aerosol pharmaceutical formulations as will be understood by those skilled in the art including, but not limited to, chlorofluorocarbon and hydrofluorocarbon propellants. The propellant is preferably a hydrofluorocarbon propellant selected from the group consisting of 1,1,1,2-tetrafluoroethane (HFA 134a), 1,1,1,2,3,3,3-heptafluoro-n-propane (HFA 227) and mixtures thereof. In some embodiments, the propellant is a single propellant selected from HFA 134a and

HFA 227. In other embodiments, the propellant is HFA 134a. While chlorofluorocarbon propellants may be utilized in aerosol pharmaceutical formulations according to the present invention, it is desirable that the formulations of the invention contain no components which may provoke the degradation of stratospheric ozone. In particular it is desirable that the formulations are substantially free of chlorofluorocarbons such as CCl3F, CCl2F2 and CF3 CCl3. If desired the propellant may additionally contain a volatile adjuvant such as a saturated hydrocarbon, for example, propane, n-butane, isobutane, pentane and isopentane or a dialkyl ether, for example, dimethyl ether. In general, up to 50% w/w of the propellant may comprise a volatile hydrocarbon, for example 1 to 30% w/w. However, formulations which are substantially free of volatile adjuvants are preferred. In certain cases, it may be desirable to include appropriate amounts of water, which can be advantageous in modifying the dielectric properties of the propellant.

The formulations according to the present invention may optionally contain one or more further ingredients conventionally used in the art of pharmaceutical aerosol formulation. Such optional ingredients include, but are not limited to, taste masking agents, sugars, buffers, antioxidants, water and chemical stabilisers.

Polar adjuvants which may, if desired, be incorporated into the formulations according to the present invention include, for example, C₂₋₆aliphatic alcohols and polyols such as ethanol, isopropanol and propylene glycol and mixtures thereof. Preferably, ethanol will be employed. In general only small quantities (e.g. 0.05 to 3.0% w/w) of polar adjuvants are required and the use of quantities in excess of 5% w/w may disadvantageously tend to dissolve the medicament. Formulations preferably contain less than 1% w/w, for example, about 0.1% w/w of polar adjuvant. Polarity may be determined, for example, by the method described in European Patent Application Publication No. 0327777. In some embodiments, it is desirable that the formulations of the invention are substantially free of polar adjuvants. “Substantially free” will generally be understood to mean containing less than 0 01% especially less than 0 0001% based on weight of formulation.

The pharmaceutical formulation may include a suitable surfactant. However, it is preferable that the formulations of the invention are substantially free of surfactant.

The formulations for use in the invention may be prepared by dispersal of the medicament in the selected propellant in an appropriate container, for example, with the aid of sonication or a high-shear mixer. The process is desirably carried out under controlled humidity conditions.

According to some embodiments of the present invention, a method of making a medicament dispenser includes filling a sealed container, such as the sealed container 20, with an aerosol pharmaceutical formulation to provide a medicament dispenser. The filling operation may be performed utilizing conventional bulk manufacturing methods and machinery well known to those skilled in the art of pharmaceutical aerosol manufacture for the preparation of large scale batches for the commercial production of filled canisters. The particulate medicament is added to a charge vessel and liquefied propellant is pressure filled through the charge vessel into a manufacturing vessel, together with liquefied propellant containing the surfactant. The drug suspension is mixed before recirculation to a filling machine and an aliquot of the drug suspension is then filled through the metering valve into the sealed container.

In an alternative process, an aliquot of the liquefied formulation is added to an open container under conditions which are sufficiently cold such that the formulation does not vaporise, and then a metering valve crimped onto the canister.

Typically, in batches prepared for pharmaceutical use, each filled canister is check-weighed, coded with a batch number and packed into a tray for storage before release testing.

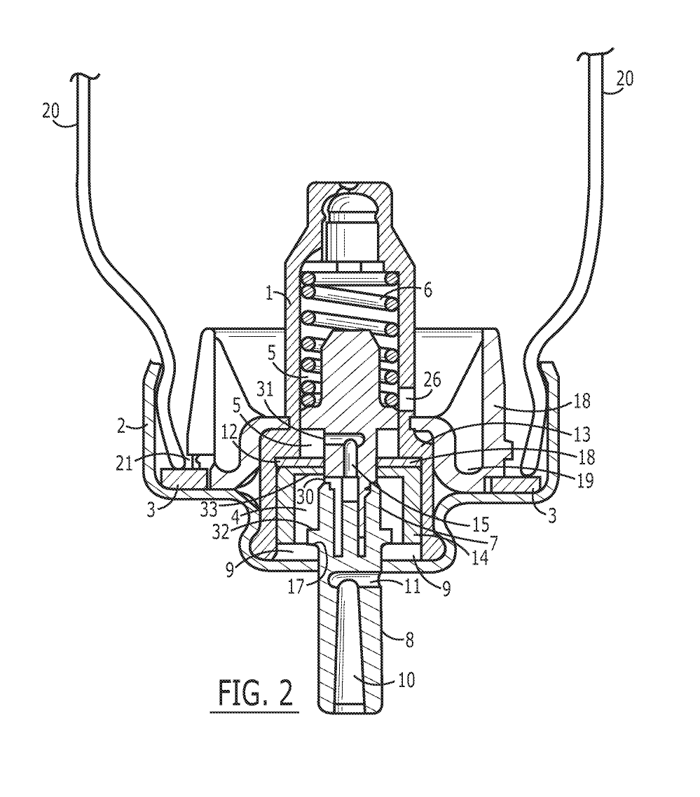

Referring now to FIG. 2, a sectional view taken along line I-I illustrated in FIG. 1 of a lower portion of a sealed container according to the present invention will be described. A cap 2 covers the open end of a container 20. A cap seal 3 is positioned between the open end of the container 20 and the cap 2. As used herein, the term “seal” is used interchangeably with the terms “sealing gasket” or “gasket”. A valve body 1 is positioned adjacent the cap 2. The valve body 1 is formed such that its lower part defines a metering chamber 4, and its upper part defines a sampling chamber 5, which also acts as a housing for a return spring 6. The words “upper” and ”“lower” are used for the container when it is in a use orientation with the neck of the container 20 and valve at the lower end of the container which corresponds to the orientation of the valve as shown in FIG. 2. The metering chamber preferably has a volume between 10 and 100 μl, more preferably between 20 and 80 The valve body may comprise various materials as will be understood by those skilled in the art, including, but not limited to, plastic and metal materials. Inside the valve body is disposed a valve stem 7, a part 8 of which extends outside the valve through lower stem seal 9 and cap 2. The upper portion of the valve stem 7 has a diameter such that it can slide through an opening in an upper stem seal 12 and will engage the periphery of that opening sufficiently to provide a seal. The valve stem may comprise various materials as will be understood by those skilled in the art including, but not limited to, plastic and metal materials.

According to a first aspect of the present invention, the metering valve has an upper stem seal that comprises a first elastomeric material and a lower stem seal that comprises a second elastomeric material different from the first elastomeric material. The first elastomeric material may comprise various polymers as will be understood by those skilled in the art including, but not limited to, low density polyethylene, chlorobutyl, acrylonitrile butadiene rubbers, butyl rubber, a polymer of ethylene propylene diene monomer (EPDM), neoprene, or chloroprene. The second elastomeric material may comprise various polymers as will be understood by those skilled in the art including, but not limited to, low density polyethylene, chlorobutyl, acrylonitrile butadiene rubbers, butyl rubber, a polymer of ethylene propylene diene monomer (EPDM), neoprene, or chloroprene.

In some embodiments according to this aspect of the present invention, the first elastomeric material and the second elastomeric material comprise different polymers. For example, the first elastomeric material may comprise an acrylonitrile butadiene polymer while the second elastomeric material comprises an EPDM polymer.

In other embodiments according to this aspect of the present invention, the first elastomeric material and the second elastomeric material comprise the same polymer, but have different extractant profiles. For example, the first elastomeric material may comprise acrylonitrile butadiene polymer and have a first extractant profile, and the second elastomeric material may comprise acrylonitrile butadiene polymer and have a second extractant profile different from the first extractant profile.

As used herein, the term “extractant profile” includes the level of one or more extractable materials and/or the gradient of one or more extractable materials taken across the thickness of the seal. Extractable materials include various compounds typically present in elastomeric gasket materials, which compounds are capable of being extracted from the materials using an aqueous or organic solvent. Such compounds include, but are not limited to, fatty acids, antioxidants, light stabilizing compounds, rubber synthesis byproducts, and other rubber extractables. More particular examples of such compounds include, but are not limited to, nonylphenol isomers, 2,2′-methylenebis(6-tertbutyl-4-methylphenol), 2,2,4,6,6-pentamethylhept-3-ene, 3′-oxybispropanitrile, oleic acid, palmitic acid, and stearic acid. Seals having different extractant profiles may be provided by various methods as will be understood by those skilled in the art including, but not limited to, the methods described in the co-pending and co-owned U.S. provisional patent application entitled “Pharmaceutical metered dose inhaler and methods relating thereto” filed Aug. 11, 2003 and the methods described in the co-pending and co-owned U.S. provisional patent application entitled “Pharmaceutical metered dose inhaler and methods relating thereto” filed Jul. 31, 2003. In some embodiments, the level of one or more extractable materials in the seal is between a lower limit of 0.001, 0.005, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85 or 0.9 and an upper limit of 0.005, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 2, 3, 4, 5, 6, 7, 8, 9, or 10 percent (by weight of the seal). In other embodiments, the level of substantially all or all of the extractable materials in the seal is between these lower and upper limits.

In still other embodiments according to this aspect of the present invention, the first elastomeric material and the second elastomeric material comprise different polymers and have different extractant profiles.

According to another aspect of the present invention, the cap seal 3 comprises a first elastomeric material and the lower stem seal 9 and/or upper stem seal 12 comprise a second elastomeric material different from the first elastomeric material. The first elastomeric material may comprise various polymers as will be understood by those skilled in the art including, but not limited to, low density polyethylene, chlorobutyl, acrylonitrile butadiene rubbers, butyl rubber, a polymer of ethylene propylene diene monomer (EPDM), neoprene, or chloroprene. The second elastomeric material may comprise various polymers as will be understood by those skilled in the art including, but not limited to, low density polyethylene, chlorobutyl, acrylonitrile butadiene rubbers, butyl rubber, a polymer of ethylene propylene diene monomer (EPDM), neoprene, or chloroprene.

In some embodiments according to this other aspect of the present invention, the first elastomeric material and the second elastomeric material comprise different polymers. For example, the first elastomeric material may comprise an acrylonitrile butadiene polymer while the second elastomeric material comprises an EPDM polymer. As another example, the first elastomeric material may comprise a polymer having a Shore A hardness of between 45 and 95, preferably between 55 and 85, and more preferably between 60 and 80, while the second elastomeric material comprises a polymer having a Shore A hardness of between 50 and 95, preferably between 60 and 85, and more preferably between 70 and 75.

In other embodiments according to this other aspect of the present invention, the first elastomeric material and the second elastomeric material comprise the same polymer, but have different extractant profiles. For example, the first elastomeric material may comprise acrylonitrile butadiene polymer and have a first extractant profile, and the second elastomeric material may comprise acrylonitrile butadiene polymer and have a second extractant profile different from the first extractant profile.

In still other embodiments according to this other aspect of the present invention, the first elastomeric material and the second elastomeric material comprise different polymers and have different extractant profiles.

In some embodiments according to this other aspect of the present invention, the cap seal comprises the first elastomeric material and the upper stem seal and lower stem seal each comprise the second elastomeric material. In preferred embodiments, the cap seal comprises an EPDM polymer and the upper stem seal and lower stem seal each comprise a nitrile polymer, such as acrylonitrile butadiene rubber.

According to still another aspect of the present invention, the cap seal 3 comprises a first elastomeric material, the lower stem seal 9 comprises a second elastomeric material different from the first elastomeric material, and the upper stem seal 12 comprises a third elastomeric material different from the first elastomeric material and different from the second elastomeric material. The first, second, and third elastomeric materials may comprise various polymers including, but not limited to, low density polyethylene, chlorobutyl, acrylonitrile butadiene rubbers, butyl rubber, a polymer of ethylene propylene diene monomer (EPDM), neoprene, or chloroprene.

In some embodiments according to this still other aspect of the present invention, the first elastomeric material, the second elastomeric material, and the third elastomeric material each comprise a different polymer.

In other embodiments according to this still other aspect of the present invention, the first elastomeric material, the second elastomeric material, and the third elastomeric material each comprise the same polymer, but each have a different extractant profile.

In still other embodiments according to this still other aspect of the present invention, the first elastomeric material, the second elastomeric material, and the third elastomeric material each comprise a different polymer and have a different extractant profile.

Still referring to FIG. 2, the upper stem seal 12 is held in position against a step 13 formed in the valve body 1 between the lower and upper parts by a sleeve 14 which defines the metering chamber 4 between the lower stem seal 9 and upper stem seal 12. The stem part 8 is formed with an inner axial or longitudinal canal 10 opening at the outer end of the stem and in communication with a radial passage 11. The valve stem 7 has a passage 15 which, when the stem is in the inoperative position shown, provides fluid communication between the metering chamber 4 and sampling chamber 5 via orifices 30 and 31, respectively. The sampling chamber 5 is in fluid communication with the interior of the container 20 via orifice 26 formed in the side of the valve body.

The valve stem 7 is biased downwardly to the inoperative position by the return spring 6 and is provided with a shoulder 17 which abuts against the lower stem seal 9. In the inoperative position as shown in FIG. 2, the shoulder 17 abuts against the lower stem seal 9 and the radial passage 11 opens below the lower stem seal 9 so that the metering chamber 4 is isolated from the canal 10 and the pharmaceutical formulation contained within the container 20 cannot escape.

A ring 18 having a “U” shaped cross section extending in a radial direction is disposed around the valve body below orifice 26 so as to form a trough 19 around the valve body. As seen in FIG. 2, the ring is formed as a separate component having an inner annular contacting rim of a diameter suitable to provide a friction fit over the upper part of valve body 1. The ring seats against step 13 below the orifice 26. While the ring 18 is illustrated in FIG. 2 as being separate from the valve body 1, it will be understood by those skilled in the art that the ring 18 may alternatively be formed as an integrally molded part of valve body 1. In some embodiments, the valve stem, the valve body, and/or at least a portion of the metering chamber wall(s) present a surface to the pharmaceutical formulation to which the one or more medicaments in the pharmaceutical formulation are non-adherent (e.g., as described in WO99/42154, WO97/16360, and WO99/50156). For example, the metering chamber (especially when composed of a plastics material) may be surface treated so as to present a substantially fluorinated surface to the formulation. Alternatively the metering chamber (especially when composed of a plastics material) may be surface treated with a siloxane such as dimethyl siloxane. As a further alternative, the metering chamber presents a substantially fluorinated surface to the formulation by virtue of being composed of a suitable substantially fluorinated material. Suitable metering chambers and surface treatments for metering chambers are described in WO 02/51483 at page 7, line 15 to page 11, line 18, for example. Suitable valve stems and surface treatments for valve stems are described in WO 02/51483 at page 11, line 21 to page 12, line 3, for example.

To use the device illustrated in FIG. 2, the sealed container is first shaken to homogenise the suspension within the container 20. The user then depresses the valve stem 7 against the force of the spring 6. When the valve stem is depressed, the shoulder 32 on the valve stem 7 comes to rest on a surface 33 of the sleeve 14. The orifice 30 comes to lie on the side of the upper stem seal 12 remote from the metering chamber 4, thereby isolating the metering chamber 4 from the sampling chamber 5. The radial passage 11 is moved into the metering chamber 4, creating fluid communication between the metering chamber 4 and the outlet canal 10 in the valve stem 7. Thus, the metered dose being held in the metering chamber 4 can exit through the radial passage 11 and the outlet canal 10.

Releasing the valve stem 7 causes it to return to the illustrated position under the force of the spring 6. The passage 15 then once again provides fluid communication between the metering chamber 4 and the sampling chamber 5. Accordingly, at this stage, liquid pharmaceutical formulation passes under pressure from the container 20 through orifice 26, through orifice 31, through passage 15, through orifice 30, and into the metering chamber 4 to fill the metering chamber 4.

Metering valves according to embodiments of the present invention may be made by various methods as will be understood by those skilled in the art. According to some embodiments of the present invention, a method of making a metering valve includes assembling a valve body, a first stem seal that includes a first elastomeric material, a second stem seal that includes a second elastomeric material different from the first elastomeric material, and a valve stem to provide a metering valve. The valve body, first stem seal, second stem seal, and valve stem are preferably similar to or the same as those described above with reference to FIG. 2.

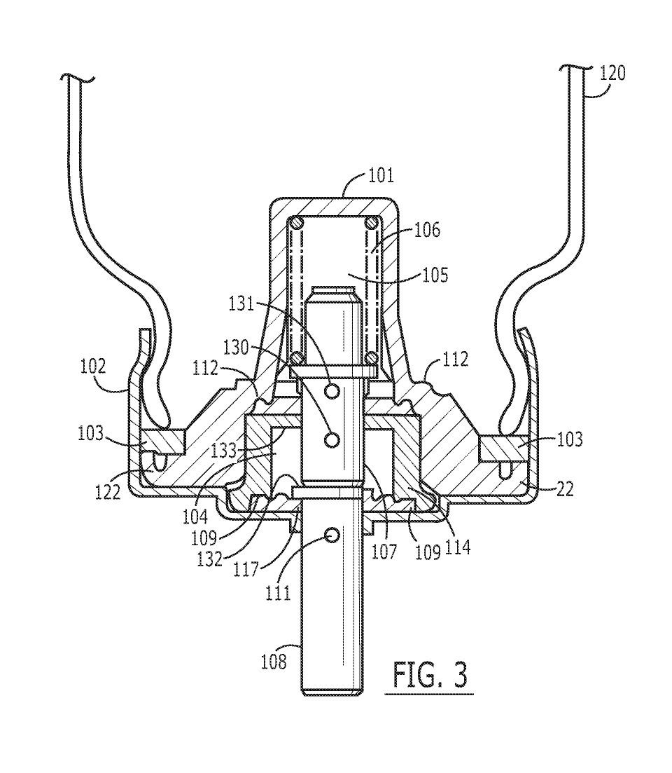

Referring now to FIG. 3, a sectional view of a lower portion of a sealed container according to the present invention will be described. The elements referred to by reference numerals 102, 103, 104, 105, 107, 108, 109, 111, 112, 114, 117, 120, 130, 131, 132, and 133 are similar to the elements referred to by reference numerals 2, 3, 4, 5, 7, 8, 9, 11, 12, 14, 17, 20, 30, 31, 32, and 33 described above in FIG. 2 and will not be further described. A valve body 101 is formed such that its lower part defines the metering chamber 104, its upper part defines the sampling chamber 105, which also acts as a housing for a resilient member 106, and a portion of the valve body 122 that supports the cap seal 103. The valve body may comprise various materials such as those described above with reference to the valve body 1 in FIG. 2. The resilient member 106 is used to bias the valve stem 107 towards the upper surface of the lower stem seal 109. The resilient member 106 may comprise various resilient members as will be understood by those skilled in the art including, but not limited to, a spring, and a flexible bushing.

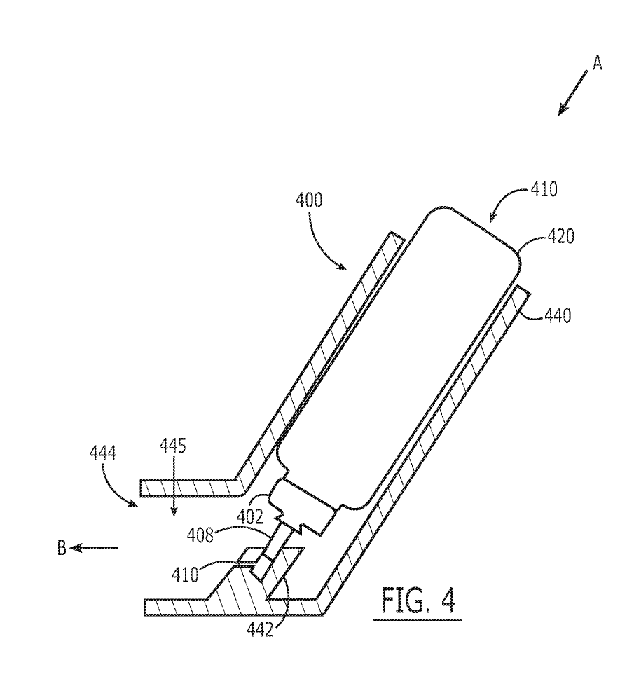

Referring now to FIG. 4, a metered dose inhaler 400 according to embodiments of the present invention will be described. The metered dose inhaler 400 includes a medicament dispenser comprising a sealed container 410 that is fitted within an actuator housing 440. The sealed container 410 includes a container 420 having an opening therein with a cap 402 covering the opening in the container 420. A metering valve having a valve stem 408 is positioned within the sealed container 410. The valve stem 408 is engaged with a nozzle block 442, which is integrally formed with the actuator housing 440. While the nozzle block 442 is illustrated in FIG. 4 as being integrally formed with the actuator housing 440, it will be understood by those skilled in the art that the nozzle block may be formed separately from the actuator housing. While the actuator housing 440 is illustrated as an oral inhalation actuator housing, it will be understood by those skilled in the art that metered dose inhalers according to the present invention may include other types of actuator housing, such as those designed for nasal administration, for example. Metered dose inhalers according to embodiments of the present invention are designed to deliver a fixed unit dosage of medicament per actuation or “puff”, for example, in the range of 2.5 to 5000 micrograms of medicament per puff, preferably in the range of from 5.0 to 2500 micrograms per puff.

MDIs taught herein may be prepared by various methods as will be understood by those skilled in the art (e.g., see Byron, above and WO196132150). According to embodiments of the present invention, a method of making a metered dose inhaler includes assembling a medicament dispenser according to the present invention, such as the medicament dispenser according to embodiments of the present invention described above, with an actuator configured to engage the medicament dispenser and dispense a pharmaceutical composition therefrom to provide the metered dose inhaler. The medicament dispenser may be made by various methods including, but not limited to, those described above with respect to embodiments of the present invention.

According to some embodiments of the present invention, a method of administering a pharmaceutical composition comprising a medicament indicated for the treatment of a respiratory disease such as asthma, rhinitis or COPD to a subject in need thereof includes actuating a metered dose inhaler according to embodiments of the present invention to administer the pharmaceutical composition to the subject. For example, referring to FIG. 4, a metered dose of the pharmaceutical formulation may be administered from the metered dose inhaler 400 by the patient placing his/her mouth over the opening in the actuator 444 and pressing the sealed container 410 into the actuator housing 440 along direction A while inhaling Pressing the sealed container 410 into the actuator housing 440 will cause the end of the valve stem 408 to engage the nozzle block, thus actuating the metering valve in the sealed container 410. A metered dose of the pharmaceutical formulation will then exit the nozzle block via orifice 443, exit the actuator via a cylindrical or cone-like passage 445 through which medicament may be delivered from the filled canister via the metering valve to the mouth of the patient along direction B and be drawn into the patients lungs.

In some embodiments, a method of treating and/or preventing the onset of a respiratory disease includes administering an effective amount of a pharmaceutical aerosol formulation to a person in need of treatment or prophylaxis of the respiratory disease, wherein the effective amount of the pharmaceutical aerosol formulation is administered from a metered dose inhaler according to embodiments of the present invention. While embodiments of the present invention have been described for treating or preventing the onset of a respiratory disease, it will be understood by those skilled in the art that method of the present invention could be used to treat or prevent any of the various disease or condition for which the medicaments described above with reference to embodiments of the medicament dispenser are indicated.

Administration of medicament in a container or MDI in accordance with embodiments of the present invention may be indicated for the treatment of mild, moderate, severe acute or chronic symptoms or for prophylactic treatment. It will be appreciated that the precise dose administered will depend on the age and condition of the patient, the particular particulate medicament used and the frequency of administration and will ultimately be at the discretion of the attendant physician. When combinations of medicaments are employed the dose of each component of the combination will in general be that employed for each component when used alone. Typically, administration may be one or more times, for example, from 1 to 8 times per day, giving for example 1, 2, 3 or 4 puffs each time.

Suitable daily doses, may be, for example, in the range 50 to 200 micrograms of salmeterol or 50 to 2000 micrograms of fluticasone propionate, depending on the severity of the disease. Thus, for example, each valve actuation may deliver 25 micrograms of salmeterol or 25, 50, 125 or 250 micrograms of fluticasone propionate. An appropriate dosing regime for other medicaments will be known or readily available to persons skilled in the art. Typically each filled canister for use in a metered dose inhaler contains 60, 100, 120, 160 or 240 metered doses or puffs of medicament.

According to still other embodiments of the present invention, a drug product includes a metered dose inhaler according to embodiments of the present invention and a packaging material forming an enclosed volume that contains the metered dose inhaler

The packaging material may be various packaging material as will be understood by those skilled in the art including, but not limited to, cartons and flexible wrappers. In some embodiments, the packaging material is a flexible wrapper that comprises a material that is substantially impermeable to ingress of atmospheric moisture and, optionally, substantially permeable to egress of propellant gas (e.g., as described in U.S. Pat. Nos. 6,119,853, 6,179,118, 6,315,112, 6,352,152, and 6,390,291). Preferably, the package will also contain within it a desiccant material as will be understood by those skilled in the art. The desiccant material may be inside the MDI and/or outside the MDI.

According to yet other embodiments of the present invention, a method of making a drug product includes packaging a metered dose inhaler according to embodiments of the present invention within a packaging material to provide the drug product. The packaging operation may be performed by various processes as will be understood by those skilled in the art, including but not limited to, those described in U.S. Pat. Nos. 6,119,853, 6,179,118, 6,315,112, 6,352,152, and 6,390,291.

It has been found that the absolute FPM measurements (before or after storage) are higher in a medicament dispenser (and/or an MDI) according to embodiments of the present invention than in a conventional medicament dispenser (and/or an MDI), which utilizes the same elastomeric material for the can seal and the one or more stem seals. Without being bound by any particular theory, it is, at the time of filing, hypothesised that embodiments of the present invention provide advantageous stabilisation of the aerosol formulation by one or more of the following effects: reducing drug deposition, improving stability of FPM even after storage, decreasing the increase in mean mass aerodynamic diameter (MMAD) during storage, and/or decreasing the GSD (Geometric Standard Deviation).

In a further aspect, embodiments of the invention provide a method of prolonging the shelf-life of a metered dose inhaler comprising assembling a metered dose inhaler that includes a medicament dispenser according to embodiments of the present invention described above to provide a metered dose inhaler having a shelf-life that is longer than the shelf-life of a conventional metered dose inhaler that includes a cap seal and a stem seal that comprises the same elastomeric material. In some embodiments, the shelf-life is measured by determining the FPM of the pharmaceutical formulation after storage under conditions such as 25, 30 or 40° C. and 50, 60, 75, or 85% relative humidity (RH) (preferred conditions are 25° C./60% RH, 25° C./75% RH, 30° C./50% RH, 30° C./60% RH, 40° C./75% RH, or 40° C./85% RH) for a time period such as 1, 4, 12, 26, or 52 weeks and comparing the determined FPM to the initial FPM. In these embodiments, the shelf life will be longer if, at the same or similar storage conditions, it takes a longer time period before the determined FPM reaches a given level. For example, if a conventional MDI exhibits a drop in FPM of 20% after storage at 20° C./75% RH for 4 weeks and an MDI of the present invention exhibits a drop in FPM of 20% after storage at 20° C./75% RH for 26 weeks, the MDI of the present invention will have a prolonged shelf-life. In some embodiments, the shelf-life is prolonged by at least 1, 2, 4, 8, or 12 weeks.

According to other embodiments of the present invention, a medicament dispenser comprising a particulate medicament, such as the medicament dispensers according to embodiments of the present invention described above, is provided in which the FPM of the particulate medicament is maintained within 15%, more preferably within 10% and especially within 5% of its original level after 4, 8, and preferably 12 weeks storage at 40° C. and 75% relative humidity.

The chemical and physical stability and the pharmaceutical acceptability of the aerosol formulations according to the invention may be determined by techniques well known to those skilled in the art. Thus the chemical stability of the components may be determined by HPLC assay, for example, after prolonged storage of the product. Physical stability data may be gained from other conventional analytical techniques such as by leak testing, by valve delivery assay (average shot weights per actuation), by dose reproducibility assay (active ingredient per actuation) and spray distribution analysis.

The suspension stability of the aerosol formulations according to the invention may be measured by conventional techniques, for example, by measuring flocculation size distribution using a back light scattering instrument or by measuring aerodynamic particle size distribution by cascade impaction, next generation impactor, multistage liquid impinger, or by the “twin impinger” analytical process. As used herein reference to the “twin impinger” assay means “Determination of the deposition of the emitted dose in pressurised inhalations using apparatus A” as defined in British Pharmacopaeia 1988, pages A204-207, Appendix XVII C. Such techniques enable the “respirable fraction” of the aerosol formulations to be calculated. One method used to calculate the “respirable fraction” is by reference to “fine particle fraction” which is the amount of active ingredient collected in the lower impingement chamber per actuation expressed as a percentage of the total amount of active ingredient delivered per actuation using the twin impinger method described above. As discussed above, the absolute “fine particle mass” (FPM) is an important parameter in relation to the present invention. The FPM may be assessed using the same apparatus as the fine particle fraction.

According to other embodiments of the present invention, a method of distributing a sealed container includes transporting a sealed container according to embodiments of the present invention described above over a distance of at least 1 yard (or 1 meter), preferably at least 1 mile (or 1 kilometer). The transporting operation can be performed via various processes as will be understood by those skilled in the art including, but not limited to, transporting via air carrier and/or transporting via ground carrier.

While some of the embodiments of the present invention described herein have focused on devices and/or methods useful for delivery of a medicament to the lungs of a patient, it will be understood by those skilled in the art that the present invention is also useful for delivery of a medicament to the nasal passages of a patient (e.g., where the medicament dispenser includes a nasal actuator instead of a mouth actuator as shown in FIG. 4).

Except as otherwise noted, all references including patent and published patent applications referred to herein are incorporated herein by reference in their entireties.

The invention will now be described further with reference the following Examples which serve to illustrate the invention but are not intended to be limiting.

### EXAMPLES

### Example I

Sealed containers including an 8 ml aluminium canister (manufactured by Presspart Inc., of Cary, N.C.) coated with a PTFE-PES coating supplied by CCL Container of Harrisonburg, Va., a neck (or cap) seal, a cap (or ferule) and a DF60 Mk42 metering valve, item no. 803309, (manufactured by Valois Pharm, of Le Vaudreuil, France) having a lower stem seal and an upper stem seal were assembled using conventional techniques known in the art. The materials used for the neck seal, the lower stem seal, and the upper stem seal in each of the sealed containers were varied according to the following matrix.

| Sealed container | Neck Seal | Lower Stem Seal | Upper Stem Seal |
|---|---|---|---|
| 1* | EPDM | EPDM | EPDM |
| 2 | EPDM | EPDM | Nitrile |
| 3 | EPDM | Nitrile | EPDM |
| 4 | EPDM | Nitrile | Nitrile |
| 5 | Nitrile | EPDM | EPDM |
| 6 | Nitrile | EPDM | Nitrile |
| 7 | Nitrile | Nitrile | EPDM |
| 8* | Nitrile | Nitrile | Nitrile |

*For comparative purposes only. Not part of the present invention.

The EPDM seals were model no. 808TS1 and/or 808TS1 EX2 seals obtained from Valois Pharm and had been extracted with ethanol. The nitrile seals were acrylonitrile butadiene rubber seals, model no. 403B and/or 404B, obtained from Valois Pharm.

The sealed containers were then filled through the metering valve with a pharmaceutical formulation containing 8 mg fluticasone propionate and 5.8 mg salmeterol xinafoate in 12 grams of 134a propellant. After filling, the sealed containers were fired and the initial fine particle mass (FPM) of the formulation was determined for each container using Anderson Cascade Impaction, with the FPM being the sum of the 3, 4, and 5 stage values.

After determining the initial FPM, the sealed containers were stored at 40° C./75% RH for 12 weeks. The FPM was then determined again using the procedure described above. The relative FPM results (with variability) are illustrated in Chart 1 below:

As can be seen in Chart 1, sealed containers 2, 3, and 4 having a neck seal made of EPDM and at least one stem seal made of nitrile exhibited improved stability (e.g., lower drops in FPM after storage) when compared to the conventional sealed container 8 having all nitrile seals.

### Example II

The procedures performed in Example I above were repeated using a pharmaceutical formulation similar to that used in Example I and using sealed containers similar to those used in Example I, with the exception that the valves were DF60 Mk42 metering valves, item no. 10002715, (manufactured by Valois Pharm, of Le Vaudreuil, France). The relative FPM results (with variability) are illustrated in Chart 2 below:

As can be seen in Chart 2, sealed containers 2, 3, and 4 having a neck seal made of EPDM and at least one stem seal made of nitrile exhibited improved stability (e.g., lower or no measurable drop in FPM after storage) when compared to the conventional sealed container 8 having all nitrile seals.

1-50. (canceled)

51. A method of administering a pharmaceutical formulation comprising a medicament indicated for the treatment of a respiratory disease to a subject in need thereof, said method comprising actuating a metered dose inhaler to administer the pharmaceutical formulation to the subject, the metered dose inhaler comprising:
a medicament dispenser comprising a sealed container having an aerosol pharmaceutical formulation present therein, said sealed container comprising a container having an opening therein;
a cap covering the opening in the container;
a metering valve adjacent the cap, said metering valve comprising: (1) at least one stem seal that comprises a nitrile polymer and (2) a valve stem; and
a cap seal positioned between the cap and the container to provide a sealed container configured to contain the aerosol pharmaceutical formulation, said cap seal comprising a polymer of ethylene propylene diene monomer (EPDM) and positioned so as to not come into direct contact with said valve stem;
wherein said aerosol pharmaceutical formulation consists essentially of a medicament and 1,1,1,2-tetrafluoroethane (HFA 143a) as propellant.

52. The method according to claim 51, wherein the respiratory disease is asthma.

53. The method according to claim 51, wherein the respiratory disease is COPD.

54. The method according to claim 51, wherein the medicament is albuterol sulphate.

55. The method according to claim 51, wherein the medicament is fluticasone propionate.

56. The method according to claim 51, wherein the aerosol pharmaceutical formulation is substantially free of surfactant and polar adjuvants

57. The method according to claim 51, wherein the aerosol pharmaceutical formulation is substantially free of polar adjuvants.
